(19) United States
(12) Patent Application Publication
(10) Pub. No.: US 2014/0065676 A1
(43) Pub. Date: Mar. 6, 2014

(54) STACKING NUCLEIC ACID AND METHODS FOR USE THEREOF

(71) Applicants: Uffe Vest Schneider, Valby (DK); Gorm Lisby, Vekso (DK); Nikolaj Dam Mikkelsen, Bronshoj (DK)

(72) Inventors: Uffe Vest Schneider, Valby (DK); Gorm Lisby, Vekso (DK); Nikolaj Dam Mikkelsen, Bronshoj (DK)

(73) Assignee: QUANTIBACT A/S, Hvidovre (DK)

(21) Appl. No.: 14/005,693

(22) PCT Filed: Mar. 28, 2012
(86) PCT No.: PCT/DK12/00030

§ 371 Date: Nov. 12, 2013

(30) Foreign Application Priority Data
Mar. 28, 2011 (DK) PA 2011 00224

(51) Int. Cl. C12P 19/34

(52) U.S. Cl. CPC C12P 19/34
USPC 435/91.2

(57) ABSTRACT

The present invention provides a novel modified oligonucleotide monomer useful in molecular biological techniques such as capture and/or detection of nucleic acids, amplification of nucleic acids and sequencing of nucleic acids. The modified oligonucleotide monomer comprises an intercalator that can intercalate into an antiparallel duplex from the major groove.

### BACKGROUND

Detection, amplification and sequencing of nucleic acids are pivotal methods in molecular biology, in research as well as in clinical diagnostics. Key reagents in such methods are oligonucleotides acting as primers and/or probes as well as nucleoside triphosphates acting as substrates for RNA or DNA polymerases.

Of main importance for oligonucleotides used as PCR templates, primers and probes are their sequence specificity and also their affinity for a complementary nucleic acid. These features can be modulated by factors intrinsic to the oligonucleotide and factors extrinsic to the oligonucleotide. Intrinsic factors are e.g. the length and nucleic acid sequence composition of oligonucleotides. Also the uses of non-natural nucleotides or backbone modifications are intrinsic factors. However, the number of available non-natural nucleotides and backbone units are limited. Accordingly, there is a need for oligonucleotides with novel modifications that can be used in molecular biology methods.

Patent application WO 2006/125447 describe a triplex forming monomer unit of the formula Z and demonstrated favorable characteristics of an oligonucleotide comprising a triplex forming monomer unit with regards to triplex formation with a double stranded nucleic acid. Based on the triplex forming characteristics, the inventors of the aforementioned patent application suggested using the oligonucleotide for detection, diagnosis and treatment. No details or data on such uses were provided.

Filichev at al., (Filichev V V, 2005) described the same triplex forming monomer unit as WO 2006/125447 and found stabilization of parallel duplex and parallel triplex by incorporation of the triplex forming monomer unit. Moreover, they found destabilization of Watson-Crick type RNA/DNA and DNA/DNA duplexes when triplex forming monomer units were inserted into an oligonucleotide, compared to the native oligonucleotide.

The triplex forming monomer described in WO 2006/125447 cannot be adapted for enzymatic incorporation into an oligonucleotide using a polymerase, because the monomer cannot function as substrate for a polymerase. Moreover, it has also been found that the triplex forming monomer described in WO 2006/125447 cannot function as template in transcription or replication. I.e. if a polymerase encounter the triplex forming monomer in a template, the polymerase cannot continue RNA or DNA synthesis.

### SUMMARY OF THE INVENTION

In a first aspect, the present invention provides a modified oligonucleotide monomer SNA (stacking nucleic acid) with the general structure:

X—B-L-I
wherein
X is a backbone monomer unit that can be incorporated into the backbone of an oligonucleotide or an oligonucleotide analogue,
B is a nucleobase, a pyrimidine or purine analog or a heterocyclic system containing one or more nitrogen atoms
L is a linker and
I is an intercalator comprising at least one essentially flat conjugated system

In a preferred embodiment, the SNA monomer comprises a conjugator K between B and L or between L and I:

X—B—K-L-I

X—B-L-K—I

The SNA monomers can be constructed to allow the intercalator I to intercalate into an antiparallel duplex from the major groove, when the SNA monomer is part of one of the strands of the duplex. In this way, the SNA monomer can stabilize antiparallel duplex formation and hence increase the affinity toward a complementary sequence.

The SNA monomers are useful in molecular biological techniques such as capture and/or detection of nucleic acids, amplification of nucleic acids and sequencing of nucleic acids. Hence other aspects of the invention are related to oligonucleotides comprising the monomer of the invention, monomers adapted for incorporation and uses of the monomer and oligonucleotides of the invention.

### BRIEF DESCRIPTION OF THE DRAWINGS

FIG. 1. The structure of the pdb entry 367d containing an intercalated functionalized acridine moiety.

FIG. 2. Overview of the TTAGGG trimer DNA duplex with an intercalated pyrene unit.

FIG. 3. Close-up on the intercalation site containing the pyrene unit.

FIG. 4 a)-e). Overview of the conformation obtained after 10 ns of MD at 50 K with 1-5 carbon linker connected to the thymidine in the sense strand.

FIG. 5 a)-e). Overview of the conformation obtained after 10 ns of MD at 50 K with 1-5 carbon linker connected to the thymidine in the antisense strand.

### DISCLOSURE OF THE INVENTION

SNA Monomer

In a first aspect, the present invention provides a modified oligonucleotide monomer SNA (stacking nucleic acid) with the general structure:

X—B-L-I
wherein
X is a backbone monomer unit that can be incorporated into the backbone of an oligonucleotide or an oligonucleotide analogue,
B is a nucleobase, a pyrimidine or purine analog or a heterocyclic system containing one or more nitrogen atoms
L is a linker and
I is an intercalator comprising at least one essentially flat conjugated system

In a preferred embodiment, the SNA monomer comprises a conjugator K between B and L or between L and I:

X—B—K-L-I

X—B-L-K—I

The SNA monomers can be constructed to allow the intercalator I to intercalate into an antiparallel duplex from the major groove, when the SNA monomer is part of one of the strands of the duplex. In this way, the SNA monomer can stabilize antiparallel duplex formation and hence increase the affinity toward a complementary sequence.

In one embodiment of the invention, it is an object to provide SNA monomers that allow enzymatic incorporation of the SNA monomer, and wherein L can reach from the nucleobase B into the major groove of an antiparallel duplex. By proper design of L, L can be forced to bend back, allowing I to intercalate into an antiparallel duplex. By placement of I into the antiparallel duplex, the antiparallel duplex is stabilized, but preferably the intercalator, I, does not interfere with enzymatic recognition of the oligonucleotide in which the SNA monomer is placed or with enzymatic incorporation of the SNA monomer into an oligonucleotide.

### The Linker L

The linker L preferably has a length selected from the group consisting of less than 30 angstroms, less than 25 angstroms, less than 20 angstroms, less than 19 angstroms, less than 18 angstroms, less than 17 angstroms, less than 16 angstroms and less than 15 angstroms, at least 3 angstroms, at least 4 angstroms, at least 5 angstroms, at least 6 angstroms, at least 7 angstroms, at least 8 angstroms, at least 9 angstroms, and at least 10 angstroms.

More preferably, the linker has a length between 1 and 30 angstroms, between 3 and 20 angstroms and most preferably between 5 and 15 angstroms, between 6 and 15 angstroms, between 7 and 15 angstroms, between 8 and 15 angstroms, between 9 and 15 angstroms and between 10 and 15 angstroms.

These lengths are particular favourable in terms of allowing the intercalator I to intercalate into the major groove of a duplex. I.e. when the SNA monomer of the invention is inserted into an oligonucleotide, it is preferred that that the affinity and/or specificity of the oligonucleotide toward a complementary nucleic acid is increased.

When the SNA does not comprise a conjugator and can be represented by X—B-L-I, a preferred embodiment of the linker L is:

—CH₂—O—(CH₂)ₙ—
wherein n is between 1 and 10, more preferably between 2 and 8, between 3 and 7, and most preferably n is 5 or 6.

Likewise, the linker may also be described as part of the SNA monomer, X—B-L-I, with the linker in bold: X—B—CH₂—O—(CH₂)ₙ—I

When the SNA monomer comprises a conjugator and can be represented by X—B—K-L-I, a preferred embodiment of the linker L is:

—(CH₂)ₙNHCO(CH₂)ₘCO—
wherein n is between 1 and 5 and m is between 1 and 5, such as where n is between 1 and 4 and m is between 1 and 4, n is between 1 and 3 and m is between 1 and 3 and more preferably, n is 1 and m is 2.

Likewise, the linker may again be described as part of the SNA monomer, X—B—K-L-I, with the linker in bold: X—B—K—(CH₂)ₙNHCO(CH₂)ₘCO—I

When the SNA monomer comprises a conjugator and can be represented by X—B-L-K—I, a preferred embodiment of the linker L is:

—(CH2)ₘ—O—(CH2-)ₙ
wherein m and n is each between 1 and 20, between 1 and 10 or between 1 and 5. Even more preferably, m is 1 and n is between 1 and 10, between 1 and 5 and most preferably n is 3 or 4.

Again, the linker may be described X—B—(CH2)ₘ—O—(CH2-)ₙ—K—I as part of the SNA monomer, X—B-L-K—I, with the linker in bold:

Other Linkers:

Other relevant linkers are e.g. those described by Ahmadian & Bergstrom M. (Ahmadian and Donald E. Bergstrom 2008, “5-Substituted Nucleosides in Biochemistry and Biotechnology.” In Modified Nucleosides in Biochemistry, Biotechnoloy and Medicine, P. Herdewijn, ed. Wiley-VCH, Weihheim, 2008, pp 251-276.), which is hereby incorporated by reference in its entirety.

The Position of L

When the B is a purine, the linker L is preferably linked to position 6 or 7 of the purine. Most preferred is linkage to position 7.

Likewise, when the B is a pyrimidine, the linker is preferably linked to position 5 or 6. Most preferred is linkage to position 5.

These linker positions are particular favourable, because DNA and RNA polymerases are particular tolerable to nucleobase modifications at these positions. I.e. a polymerase can often use nucleotides that are modified at the aforementioned positions as substrates for DNA or RNA synthesis. One such example is nucleotide triphosphates that have a biotin group conjugated to position 5 of a pyrimidine. Likewise, SNA triphosphates modified in these positions will be favourable in terms of being substrates for polymerases.

### The Conjugator K

As mentioned, in a preferred embodiment, the SNA monomer of the invention comprises a conjugator K. In the present context, the term conjugator means that K comprises p-orbitals that overlap with those of the intercalator or the nucleobase. K may be selected from the group consisting of alkenyl of 2 to 12 carbons, alkynyl of 2 to 25 carbons or diazo or combinations thereof with a length of no more than 25 carbons or/and nitrogen atoms as well as monocyclic aromatic ring systems.

In a preferred embodiment, K is acetylene or repetitive acetylenes.

Most preferably, K is ethynyl.

### Preferred Embodiments of K—I

In a preferred embodiment, K—I is ethynyl-aryl and preferably ethynyl aryl is 1-ethynylpyrene.

### Preferred Embodiments of K-L

A preferred embodiment of K-L is:

C≡C—(CH₂)ₙNHCO(CH₂)ₘCO
wherein n is between 1 and 5 and m is between 1 and 5, such as where n is between 1 and 4 and m is between 1 and 4, n is between 1 and 3 and m is between 1 and 3 and more preferably, n is 1 and m is 2.

Also K-L may be described as part of the SNA monomer X—B—K-L-I, with K-L in bold: X—B—C≡C—(CH₂)ₙNHCO(CH₂)ₘCO—I

### Preferred Embodiments of L-K

A preferred embodiment of L-K is:

(CH₂)ₘ—O—(CH₂)ₙ—C≡C
wherein m and n is each between 1 and 20, between 1 and 10 or between 1 and 5. Even more preferably, m is 1 and n is between 1 and 10, between 1 and 5 and most preferably n is 3 or 4.

And when described as part of the SNA monomer X—B-L-K—I, with L-K in bold:

X—B—(CH₂)ₘ—O—(CH₂)ₙ—C≡C—I

### Preferred Embodiments of B

B is preferably a pyrimidine or purine as illustrated by structures 1-20, where B is shown as part of the SNA monomer:

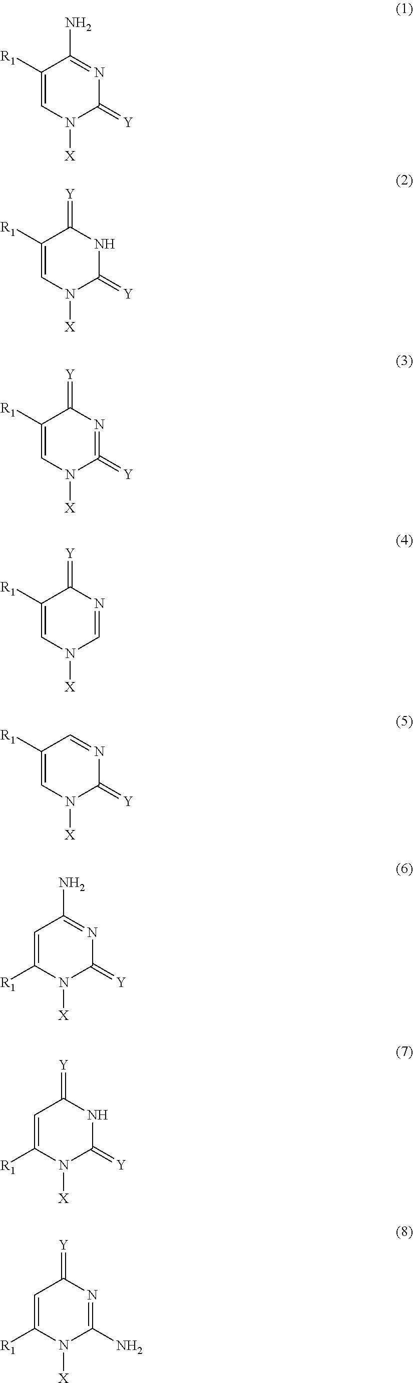

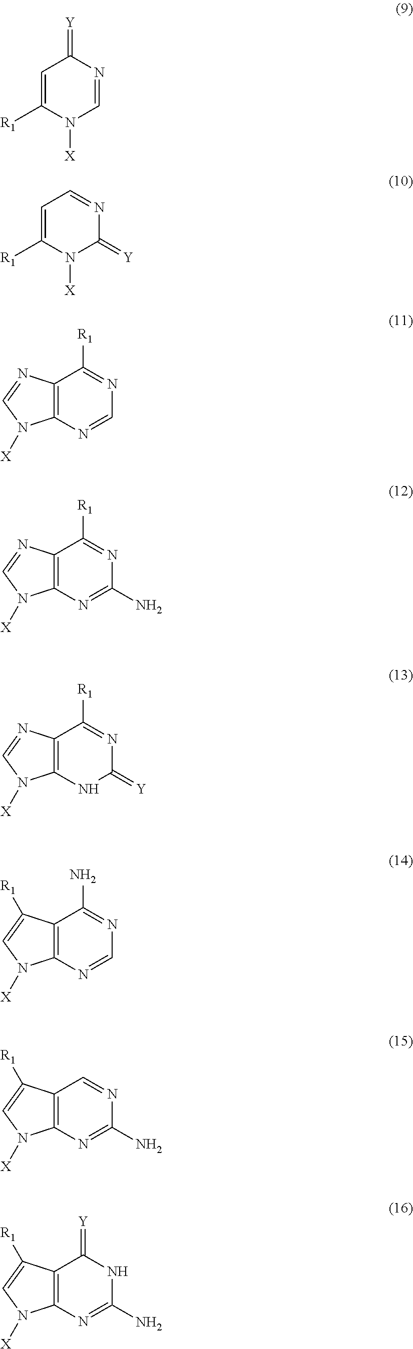

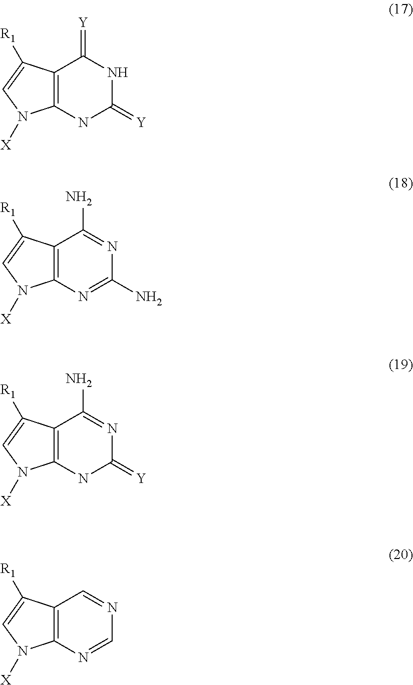

wherein
Y=O or S and
R₁ is L-I, K-L-I or L-K—I.

Particular preferred versions of L-I, K-L-I and L-K—I are described above and below.

Hence, B is preferably selected from the group of B structures illustrated in structures 1-20.

### The Intercalator I

The intercalator I of the SNA monomer of the invention comprises at least one essentially flat conjugated system, which is capable of co-stacking with nucleobases of DNA, RNA or analogues thereof.

In a preferred embodiment, I is selected from the group of bi-cyclic aromatic ringsystems, tricyclic aromatic ringsystems, tetracyclic aromatic ringsystems, pentacyclic aromatic ringsystems and heteroaromatic analogues thereof and substitutions thereof.

Particular preferred embodiments of I is pyrene, phenanthroimidazole and naphthalimide:

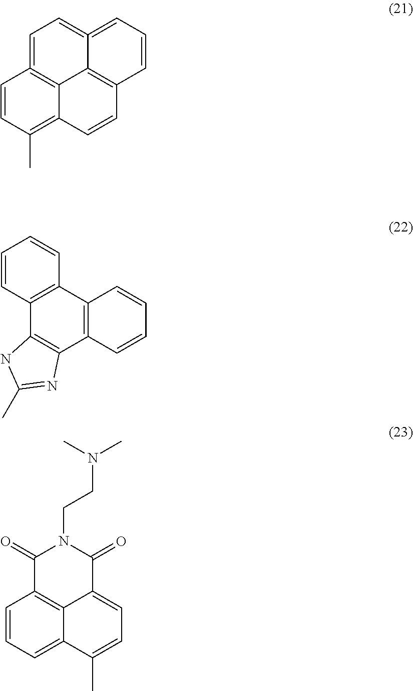

Preferred Monomers of the Invention L-K—I, K-L-I, L-I

As will be appreciated from the above description the linker L, the optional conjugator K and the intercalator I, can be combined in many ways to form favorable monomers of the invention. The synthesis of exemplary combinations is outlined in the examples section.

Second Aspect

A second aspect of the invention is an SNA monomer of the first aspect adapted for enzymatic incorporation into an oligonucleotide. In this aspect, the oligonucleotide monomer will typically be a nucleotide triphosphate.

Third Aspect

A third aspect of the invention is an SNA monomer of the first aspect adapted for incorporation into an oligonucleotide using standard oligonucleotide synthesis. In this aspect, the oligonucleotide monomer will typically be a nucleoside phosphoramidite.

Fourth Aspect

A fourth aspect of the invention is an oligonucleotide comprising the SNA monomer of the first aspect. Preferably, the (other) monomers of the oligonucleotide are either DNA or RNA monomers. The oligonucleotide may be synthesized enzymatically using the SNA monomer adapted for enzymatic incorporation into an oligonucleotide (of the second aspect of the invention) or the oligonucleotide may be synthesized using standard oligonucleotide synthesis and the SNA monomer adapted for incorporation into an oligonucleotide using standard oligonucleotide synthesis (of the third aspect of the invention).

Fifth Aspect

A fifth aspect of the invention is use of the SNA monomer adapted for enzymatic incorporation (of the second aspect of the invention) as substrate for a polymerase, e.g. in sequencing or PCR.

Sixth Aspect

A sixth aspect of the invention is use of the oligonucleotide comprising the SNA monomer (as described in the fourth aspect of the invention) as primer or template in a polymerase chain reaction (PCR).

Seventh Aspect

A seventh aspect of the invention is a method comprising the steps of
a. Providing a template nucleic acid
b. Providing a first primer oligonucleotide
c. Providing a polymerase
d. Providing a nucleotide triphosphate mixture
e. Mixing the components of steps a-d and providing conditions that allow the primer to anneal to the template.
f. Under conditions allowing primer extension, extending the first oligonucleotide annealed to the template
wherein the first primer oligonucleotide comprise a SNA monomer and/or
wherein the template nucleic acid comprise a SNA monomer and/or
wherein the nucleotide triphosphate mixture comprise a SNA monomer adapted adapted for enzymatic incorporation into an oligonucleotide (as described in the second aspect of the invention).

In a preferred embodiment, the method further comprises the steps of
g. Providing a second primer oligonucleotide, which is complementary to the first extension product of step f
h. Denaturing the product of the step f
i. Under conditions allowing primer extension, extending the second oligonucleotide annealed to the first extension product

In one embodiment, the second primer oligonucleotide comprises a SNA monomer.

### EXAMPLES

### Example 1

### A Thymine-1-Ethynylpyrene Conjugate Based on Molecular Modeling

Results and Discussion:

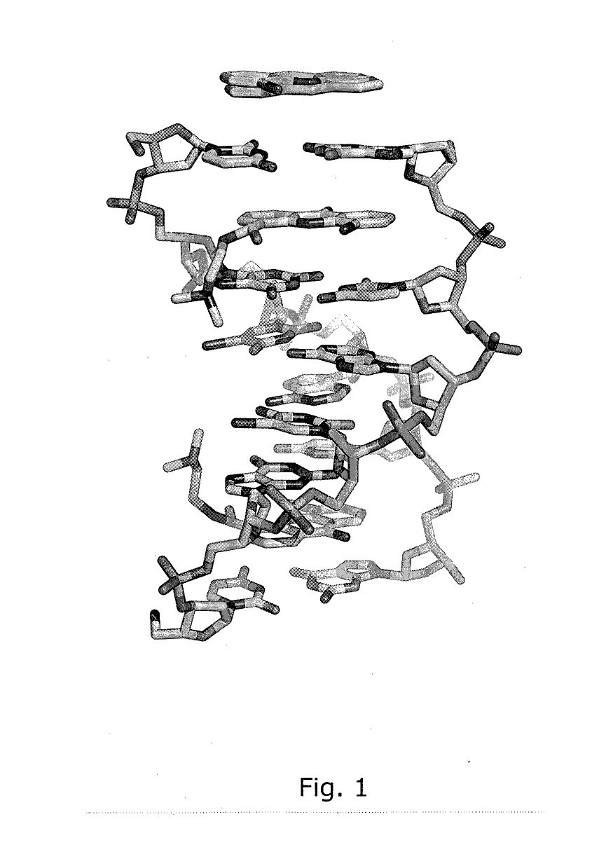

The structure of a typical intercalation between acridine and DNA was acquired from www.pdb.org (ID 367D) (A K Todd, A Adams, J H Thorpe, W A Denny, L P G Wakelin and C J Cardin, J. Med. Chem. 1999, 42, 536-540). This structure contains an intercalated acridine fragment (FIG. 1), which was used to position the pyrene moiety. To model the incorporation of the pyrene unit a DNA hexadecamer with a trifold repeat structure (TTAGGG)₃ was build in the so-called B-DNA conformation.

From these two structures a new TTAGGG trimer with a pyrene intercalated was constructed and energy minimized using molecular mechanics. The four nucleotides lining the intercalation site have been shown in bold, with the top strand designated “sense” for reference and the bottom strand “antisense”:

| | |
|---|---|
| 5′-TTAGGGTTAGGGTTAGGG-3′ | (sense strand) |
| 3′-AATCCCAATCCCAATCCC-5′ | (antisense strand) |

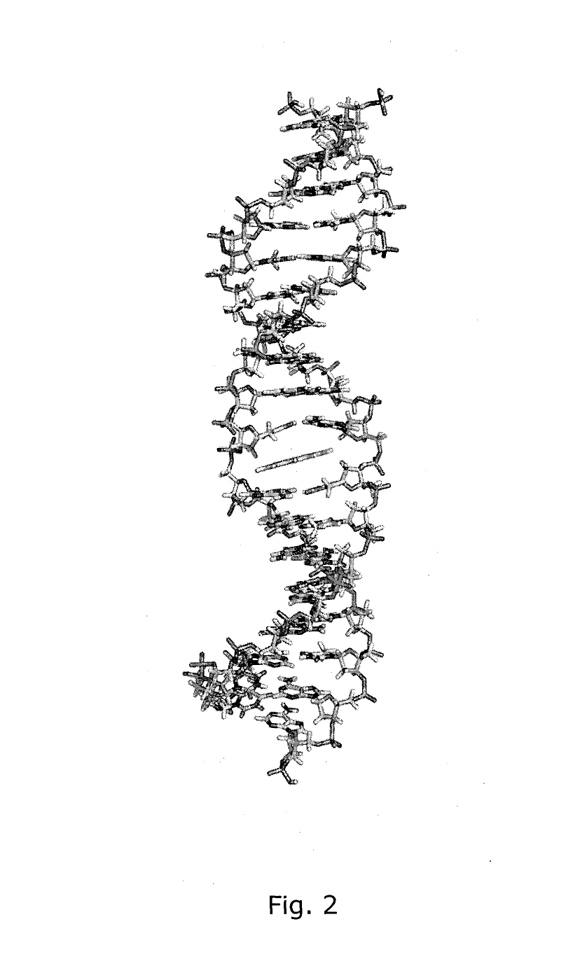

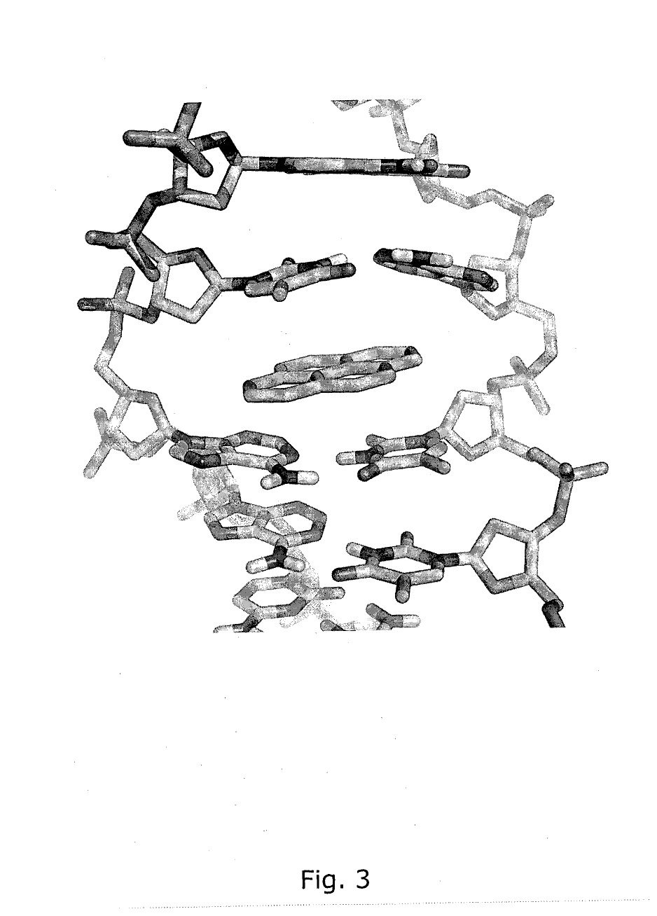

The resulting structure remained in the well-known, stabilized duplex conformation (FIG. 2), and when inspecting in detail it is clear that the all hydrogen bonds are retained (FIG. 3).

To link the pyrene unit to the DNA strand we envision that a variant of thymine with a CH₂OH instead of the methyl group, 5-(hydroxymethyl)uracil, could be used as starting point. The pyrene should still contain an alkyne group, thus we built new structures having 1 to 5 carbon atoms in the linker between the alkyne-pyrene unit and the oxygen of the nucleobase). Due to the inherent chirality of the structure there is a difference in length depending on whether the attachment is constructed to the thymidine in the sense strand (below pyrene in FIG. 3) or in the antisense strand (above pyrene in FIG. 3). To allow the structures to avoid unfavorable interactions introduced during the manual building of the constructs a series of short molecular dynamics (MD) simulation was carried out. The simulations were run for 10 ps with a temperature set to 50 K, 100 K, 150 K, 200 K, 250 K and 300 K. All the structures showed considerable deviations from the initial helical geometry at higher temperatures, thus we have selected to use structures obtained after simulations at 50 K.

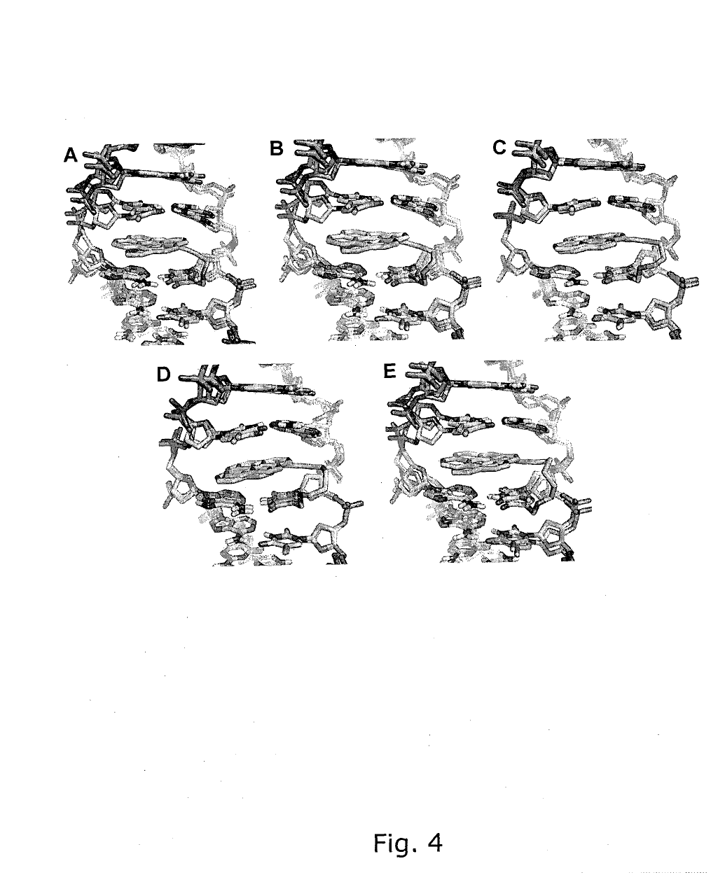

FIG. 4 show an overlay of the intercalation site between the unlinked pyrene unit and the linked pyrene unit using a spacer of 1 to 5 carbons (FIG. 4 a-e) with the modified nucleobase in the sense strand. We have chosen to use a superposition of the 8 nucleotides closest to the intercalation site in our inspection of the structures to avoid the influence of changes in more remote regions of the helix.

From these structures it is evident that both the 3-carbon and the 4-carbon linker (n=3 and n=4, FIG. 3) are capable of achieving an unstrained geometry where the unlinked and the linked pyrene units are superimposable. A three- or four-methylene spacer thus appears to be optimal for intercalation of a conjugate thymidine in the sense strand.

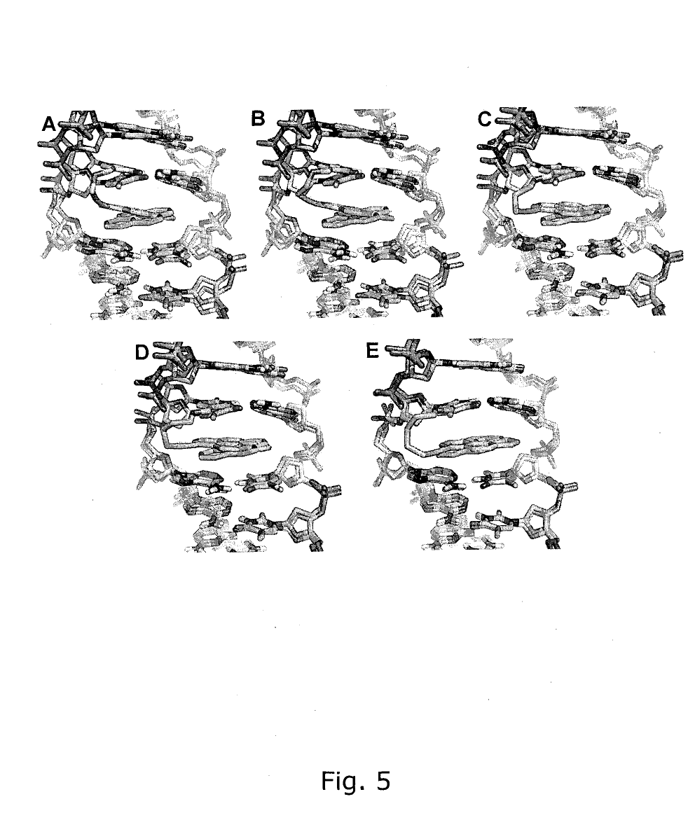

In a similar fashion we have created a link from the thymine “above” the pyrene unit (with the modified nucleobase in the antisense strand) and obtained the following structures (FIG. 5 a-e).

When using the thymine located “above” the pyrene unit none of the linkers were capable of achieving a fully unstrained geometry. The longest 5-carbon chain used in the study seems to be best at accommodating the 180° turn necessary in order to connect the oxygen of the functionalized thymine with the alkynyl linker.

The modeling data described above suggests that the ideal construct would be a 3- or 4-methylene spacer between the ethynylpyrene and (5-hydroxymethyl)uracil, incorporated in an oligonucleotide in the place of thymine in the sense strand (see above).

Synthesis

A possible synthesis of the 1-ethynylpyrene-nucleotide conjugate with a 4-carbon spacer is outlined in Scheme 1.

Commercially available 5-(hydroxymethyl)uracil can be alkylated with hex-5-yn-1-ol (also commercially available) under acidic conditions (M S Motawia, A E-S Abdel-Megied, E B Pedersen, C M Nielsen and P Ebbesen, Acta Chem. Scand. 1992, 46, 77-81; A E-S Abdel-Megied, E B Pedersen and C Nielsen, Monatshefte Chem. 1998, 129, 99-109) and a Sonogashira coupling (K Sonogashira, Y Tohda and N Hagishara, Tetrahedron Lett. 1975, 16, 4467-4470) with 1-bromopyrene introduces the intercalator. Bis-silylation of the pyrimidinedione sets it up for a glycosylation of 2-deoxy ribose triacetate mediated by TMSOTf (M S Motawia, A E-S Abdel-Megied, E B Pedersen, C M Nielsen and P Ebbesen, Acta Chem. Scand. 1992, 46, 77-81; A E-S Abdel-Megied, E B Pedersen and C Nielsen, Monatshefte Chem. 1998, 129, 99-109). After separation of the β- from the undesired α-anomer, the two acetyl groups can be removed, followed by introduction of the DMT group for protection of the primary alcohol and activation of the 3′-position as the phosphoamidate.

The proposed synthetic route is 7 steps overall, which should be a manageable task.

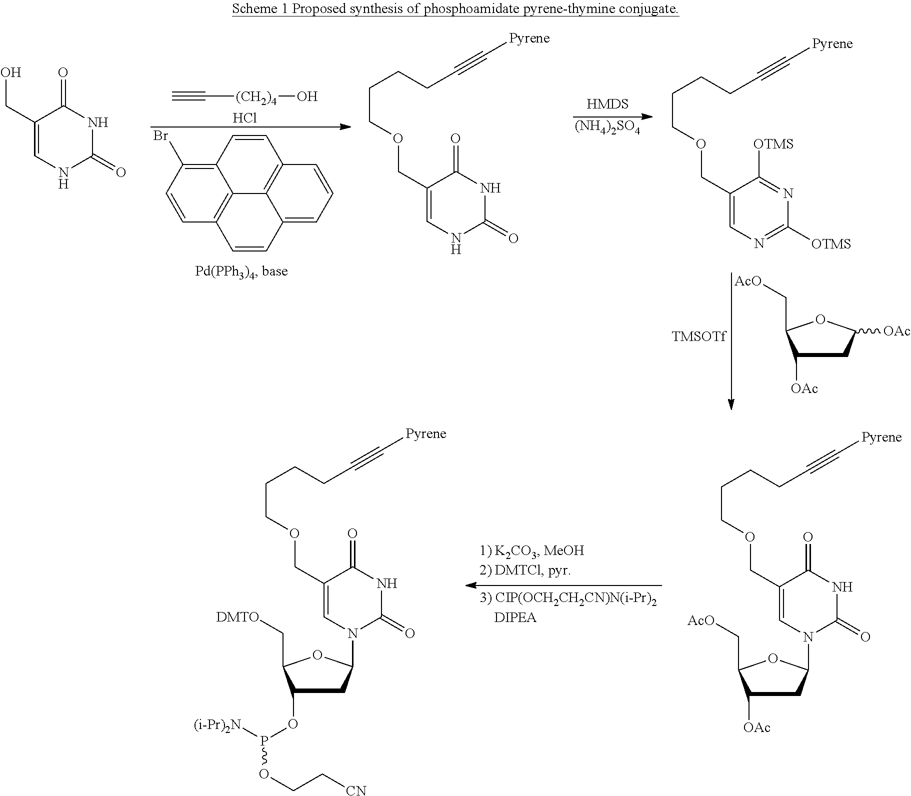

### CONCLUSION

Modeling studies of a short (18 bp) DNA double helix with an intercalating pyrene have shown that the best design for a duplex with the pyrene unit conjugated to a modified thymine base is a simple 3- or 4-carbon spacer attached to 1-ethynylpyrene in the sense strand. Furthermore, a 7-step synthetic route that will provide a phosphoamidate for incorporation in an oligonucleotide with a 4-carbon spacer between a modified thymine base and the pyrene has been outlined.

### Example 2

### Synthesis of Other Exemplary Monomers of the Invention

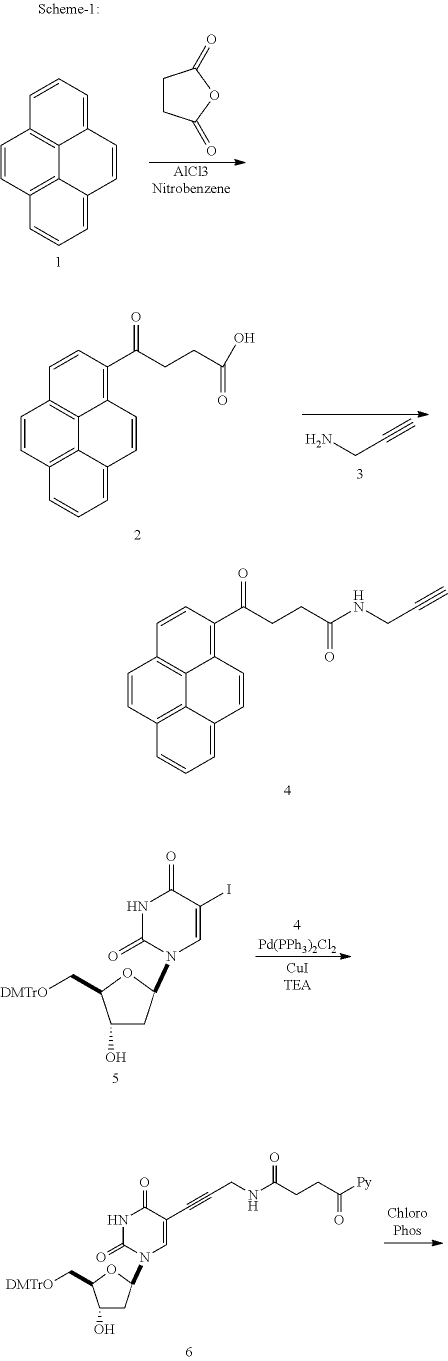

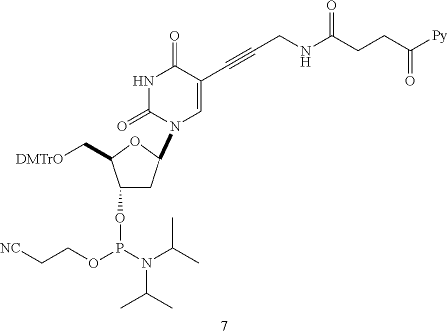

Stage-1:

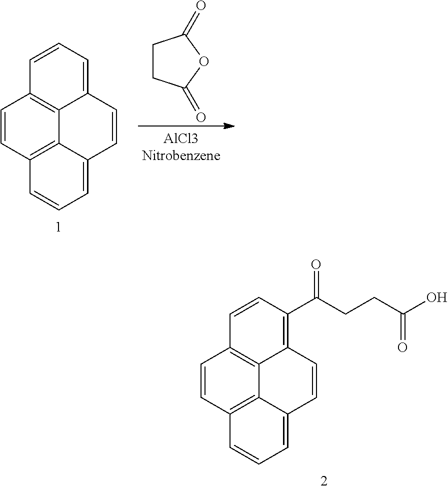

### 4-oxo-4(pyrene-1-yl)-butyric acid (2)

AlCl₃ (26.6 g, 199.86 m·moles) was added to the stirred solution of succinic anhydride (10 g, 99.93 mmol) in nitrobenzene (1000 mL) at 0° C. and followed by compound-1 (20.2 g, 99.93 mmol) was added at same temperature, then the reaction mixture was stirred at room temperature for 18 h. The progress of reaction was monitored by TLC; TLC shows the complete disappearance of starting material. The reaction mixture was poured in to 600 ml of 25% ice cold hydrochloric acid solution. Filtered the yellow colored solid compound and dried completely. The product crystallized from EtOH, to furnish compound-2 (21.8 g, 72%) as yellow colored solid.

Stage-2:

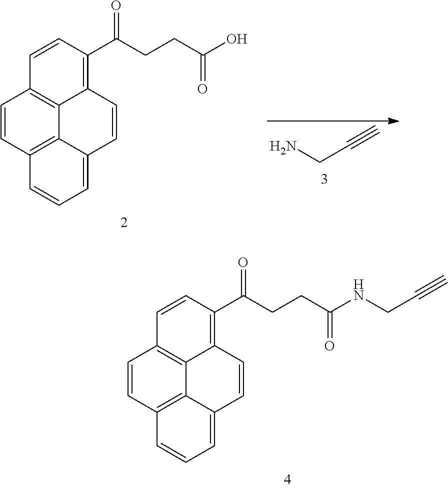

### N-Propyl-oxo-pyrene butyric acid amide (4)

DIPEA (18.6 mL, 132.48 mmol) was added to the stirred solution of compound-2 (10 g, 33.11 mmol) in dry DMF (70 ml) and 1, 2-Dichloroethane (50 mL) at room temperature under nitrogen atmosphere. Then the reaction mixture was cooled at 0° C., then lot wise added EDC.HCl (6.3 g, 33.11 mmol) and followed by HOBt (5.1 g, 33.11 mmol) under nitrogen atmosphere. Compound-3 (2.3 mL, 33.11 mmol) was added drop wise to the above mixture at 0° C. under nitrogen atmosphere. Then the reaction mixture was stirred at room temperature for 5 h. The progress of the reaction was monitored by TLC, starting material was disappeared. Then 500 ml of water was added to the reaction mixture to precipitate the product. The precipitate was filtered and the solid compound was washed with 20% Ethyl acetate in Hexane. The yellow colored solid compound was dried over P₂O₅ to furnish compound-4 (7.1 g, 63%) as yellow colored solid.

Stage-3:

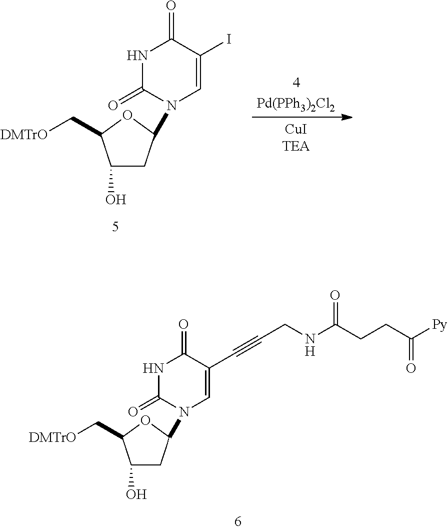

Pyrene-oxo amide dU (6):

Compound-4 (3.9 g, 11.43 mmol) was added to the stirred solution of compound-5 (5 g, 7.62 mmol) in dry THF (100 ml) at room temperature under nitrogen atmosphere, and triethylamine (4.3 mL, 30.48 mmol) was added. Then the solution was degassed by sparging with nitrogen gas for 30 minutes, Pd (PPh₃)₂Cl₂ (535 mg, 0.762 mmol) was added and again degassed for 15 min, finally added CuI (72 mg, 0.381 mmol), the reaction mixture was stirred at room temperature for 2 h. The reaction mixture was filtered through celite pad, the filtration was evaporated under reduced pressure and the compound was dissolved in DCM and washed with water and brine solution. The organic layer was dried under Na₂SO₄, filtered, evaporated under reduced pressure. The crude compound was purified by using silica gel column chromatography (60-120 mesh, 50-60% EtOAc in Hexane) to get yellow colored solid compound-6 (5.5 g, 83%).

Stage-4:

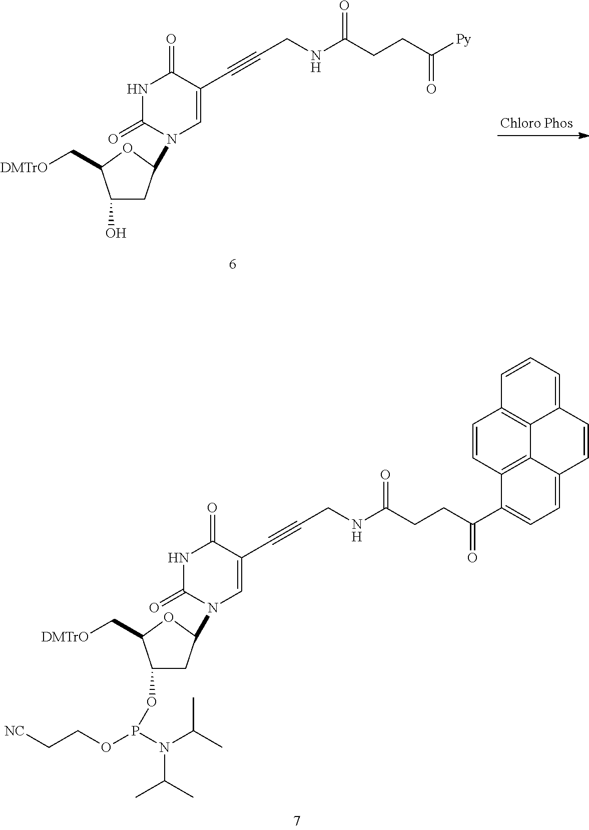

### Pyrene-oxo-5′-DMT-amidite dU (7)

Compound-6 (1.2 g, 1.38 mmol) was co-evaporated two times with dry toluene under nitrogen atmosphere and dried under high Vacuum pressure, desolved in 20 ml of dry DCM and added 1-H-tetrazole (126 mg, 1.79 mmol), followed by Phos reagent (0.6 mL, 1.79 mmol) under nitrogen atmosphere at room temperature. The reaction was stirred at room temperature for 3 h, and then precipitated with DCM/Hexane two times; finally the viscous solid compound was dissolved in DCM and evaporated under rotavapor, dried under high vacuum to get compound-7 (850 mg, 61%) as pale colored solid.

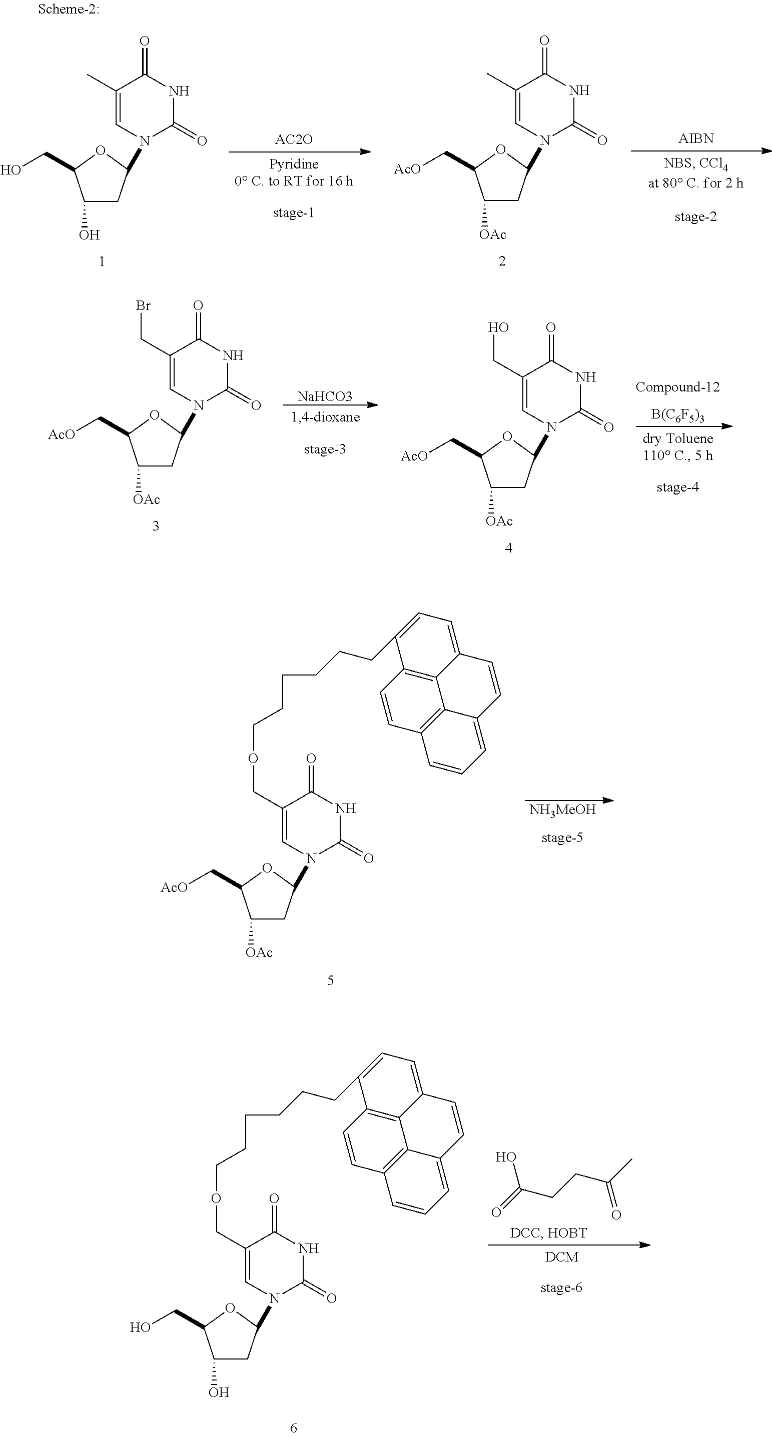

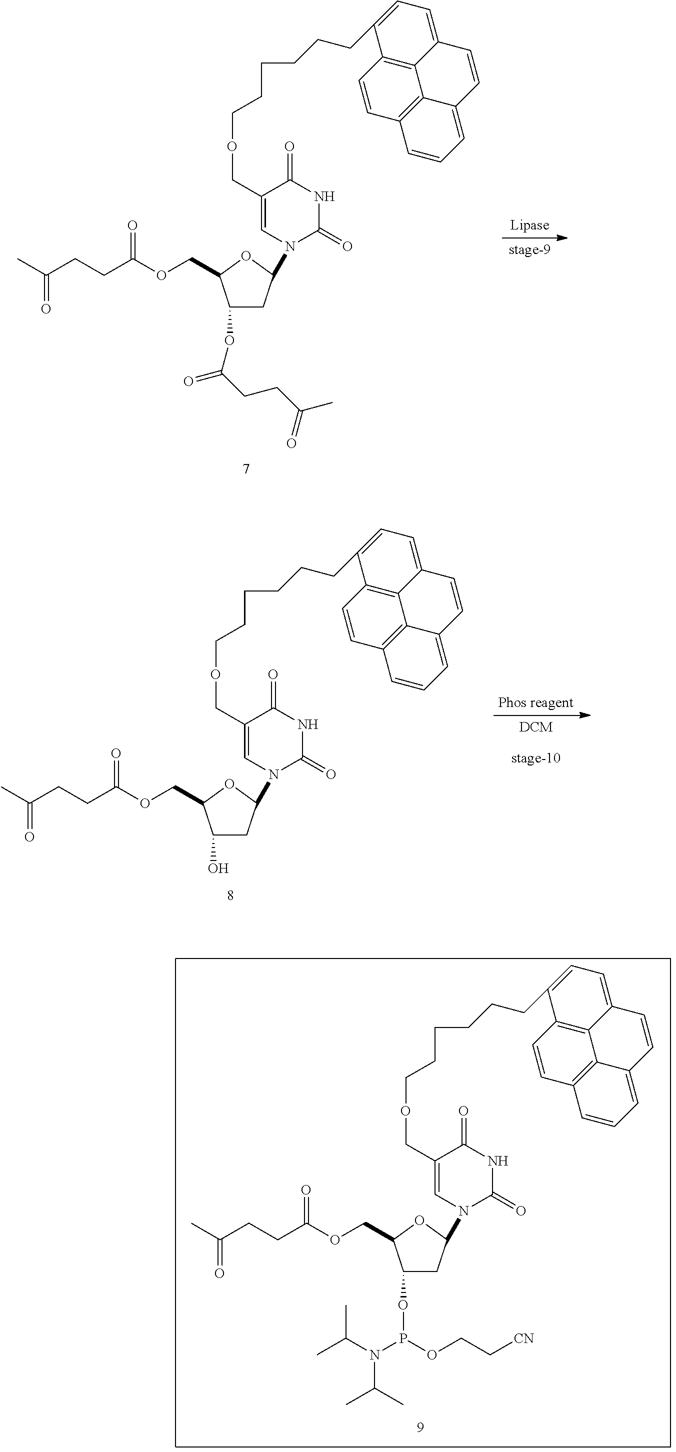 [SMILES: CC(=O)CCC(=O)OC[C@H]1O[C@@H](n2cc(COCCCCCCc3ccc4ccc5cccc6ccc3c4c56)c(=O)[nH]c2=O)C[C@@H]1OC(=O)CCC(C)=O ; CC(=O)CCC(=O)OC[C@H]1O[C@@H](n2cc(COCCCCCCc3ccc4ccc5cccc6ccc3c4c56)c(=O)[nH]c2=O)C[C@@H]1O ; CC(=O)CCC(=O)OC[C@H]1O[C@@H](n2cc(COCCCCCCc3ccc4ccc5cccc6ccc3c4c56)c(=O)[nH]c2=O)C[C@@H]1OP(OCCC#N)N(C(C)C)C(C)C]

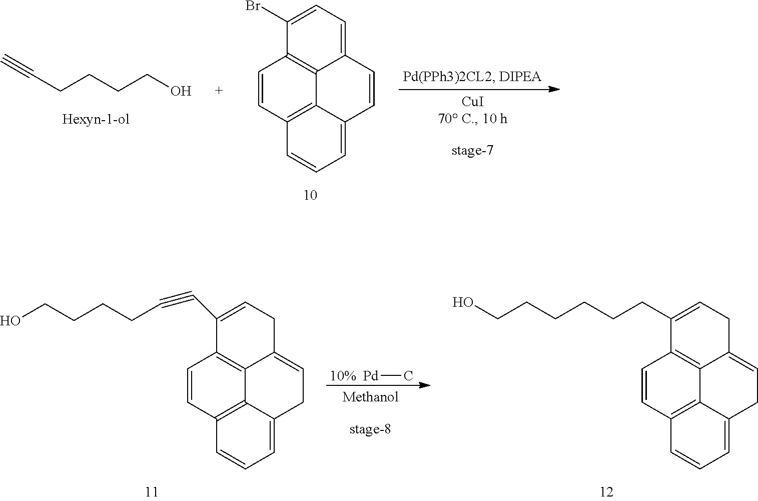

Stage-1:

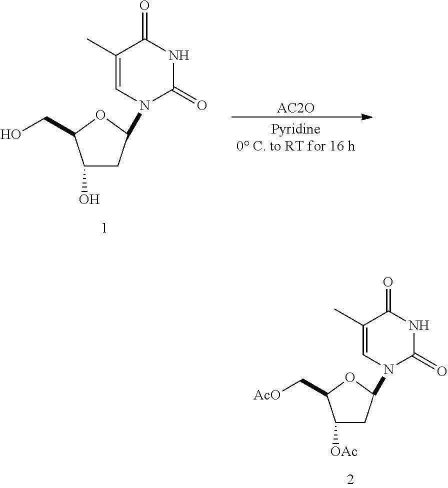

### 5′,3′-diacetyl-dT (2)

To a solution of compound-1 (100 g, 412.83 mmol) was dissolved in dry pyridine (1500 mL) and the reaction mixture was cool to 0° C. To this stirred suspension, acetic anhydride (156 mL, 1651.32 mmol) was added drop wise over a period of 15-20 minutes, under nitrogen atmosphere. The reaction mixture was stirred at room temperature for 16 h, to get a clear solution (pH was neutral). The reaction mixture was monitored by TLC (80% EtOAc/Hexane). TLC shows most of the starting material disappear. The reaction was cooled to 0° C. and quench with 206 mL of methanol. Major portion of the pyridine was removed under reduced pressure and the crude compound was dissolved in water (1000 mL) and ethyl acetate (1000 mL) and organic layer was separated, aqueous layer extracted with EtOAc (250 mL×2 times), combined organic layers wash with 2N HCl (200 mL), saturated NaHCO₃ (250 mL), water (250 mL×2 times) and brine (250 mL), dried with anhydrous Na₂SO₄ and solvent was evaporated under reduced pressure. Crude (viscous) compound was precipitated with 30% Ethyl acetate/Hexane (500 mL×2 times), to get white crystalline solid. The compound was taken in to next step with out further purification. The Product was characterized by ¹HNMR and MS.

Yield: 124 g (92%).

### 76SPL02211-02.

Stage-2&3:

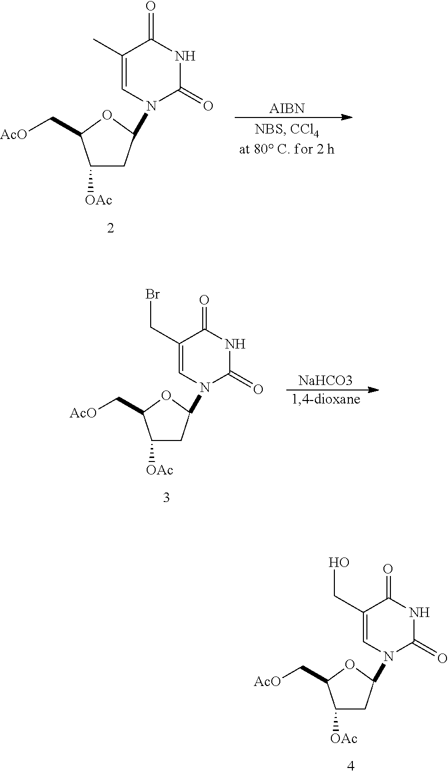

### 5-Hydroxymethyl-5′,3′-O-Diacetyl-2′-deoxyuridine (4)

Compound-2 (19 g, 58.22 mmol) was co-evaporated with anhydrous benzene 50 mL), and 300 mL of dry benzene was added. Next, reaction mixture was slowly heated to 110° C. for 10 min, under nitrogen atmosphere and NBS (12.6 g, 71.03 mmol) and AIBN (513 mg) were added to the above solution. The progress of the reaction was monitored by TLC, starting material disappeared. The reaction mixture was filtered in hot condition and evaporated solvent under reduced pressure to get compound-3 (23 g of gammy solid compound). The crude compound-3 (23 g) was dissolved in 150 mL of 1, 4-dioxane and the reaction mixture was cool to 0° C. Then NaHCO₃ (7.6 g) was dissolved in 150 mL of water, and added drop wise to the above solution at 0° C. The mixture was stirred at room temperature for 1 h. Solvent was evaporated under reduced pressure. The crude compound was purified by silica gel column chromatography (4-5% of MeOH in DCM) to furnish compound-4 (9 g, 45.2% from two steps) as pale yellow solid.

### 74 & 75 SPL02211-02.

Stage-4:

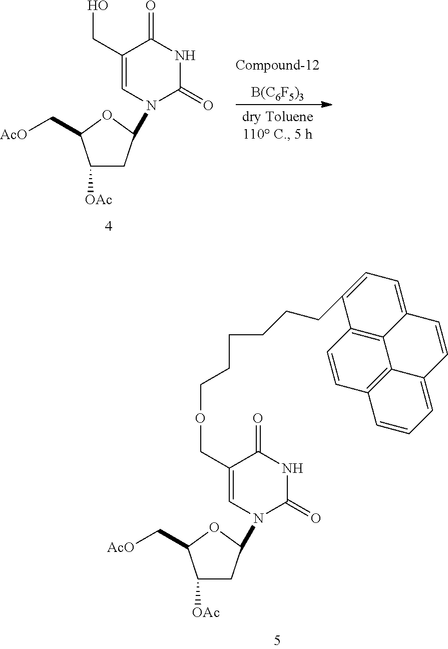 [SMILES: CC(=O)OC[C@H]1O[C@@H](n2cc(CO)c(=O)[nH]c2=O)C[C@@H]1OC(C)=O ; "B(C6F5)3" ; "Compound-12" ; CC(=O)OC[C@H]1O[C@@H](n2cc(COCCCCCCc3ccc4ccc5cccc6ccc3c4c56)c(=O)[nH]c2=O)C[C@@H]1OC(C)=O]

### 5-methylhydroxy-pyrene-hexane-5′,3′-O-Diacetyl-2′-deoxyuridine (5)

To a solution of compound-4 (3.0 g, 8.77 mmol) and compound-12 (2.1 g, 7.01 mmol) was dissolved in dry toluene at room temperature under nitrogen atmosphere. Then B(C₆F₅)₃ (449 mg, 0.87 mmol) was added to the reaction mixture under nitrogen atmosphere, Then the mixture was refluxed at 110° C. for 5 hrs. The progress of the reaction was monitored by TLC, starting material disappeared. Then reaction mixture was cool to room temperature and evaporated under reduced pressure. The crude compound was dissolved with water (50 mL) and ethyl acetate (50 mL) and organic layer was separated, aqueous layer was extracted with EtOAc (25 mL×2 times), combined organic layers was wash with water (20 mL), brine (25 mL), dried over anhydrous Na₂SO₄ and evaporated under reduced pressure. The viscous liquid compound-5 (4.0 g) was taken for the next step. The compound was characterized by LCMS.

### 40SPL02211-03.

Stage 5:

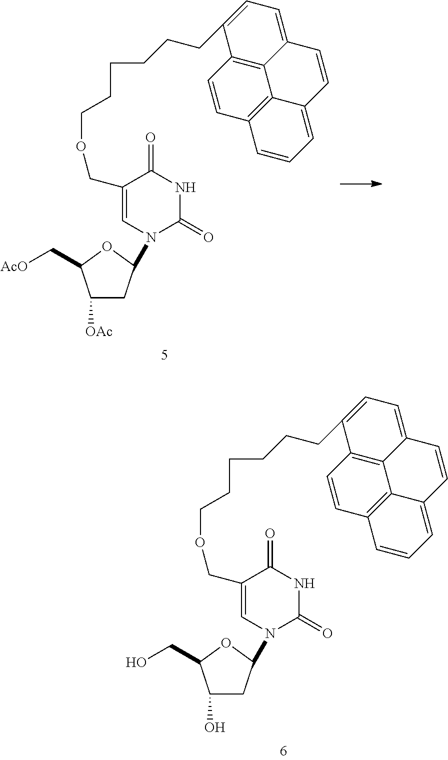 [SMILES: CC(=O)OC[C@H]1O[C@@H](n2cc(COCCCCCCc3ccc4ccc5cccc6ccc3c4c56)c(=O)[nH]c2=O)C[C@@H]1OC(C)=O ; O=c1[nH]c(=O)n([C@H]2C[C@H](O)[C@@H](CO)O2)cc1COCCCCCCc1ccc2ccc3cccc4ccc1c2c34]

### 5-methylhydroxy-pyrene-hexane-2′-deoxyuridine (6)

Compound-5 (4.0 g) was dissolved in 60 mL of MeOH.NH₃ solution, and stirred at room temperature for 16 h. The solvent was evaporated under reduced pressure, and the crude compound was diluted with EtOAc (60 mL), the organic layer was wash with water (10 mL), brine (10 mL), dried over anhydrous Na₂SO₄ and evaporated under reduced pressure. The crude compound was purified by silica gel (60-120 mesh) column chromatography, eluted with 5% MeOH in DCM to get compound-6 (410 mg, 8% from two steps) as off white solid.

### 42SPL02211-03.

Stage-6:

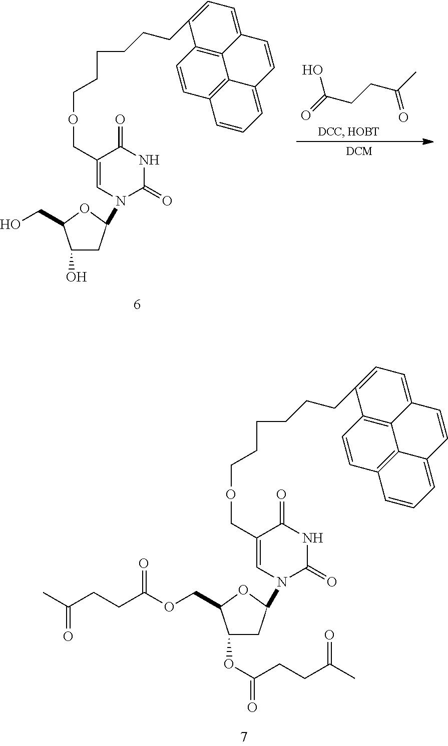

### 5-methylhydroxy-pyrene-hexane-5′,3′-O-lev 2′-deoxyuridine (7)

Compound-6 (25 mg, 0.04 mmol) was dissolved in dry DCM under nitrogen atmosphere, and cooled the solution at 0° C. Then added DCC (11 mg, 0.05 mmol), HOBt (6 mg, 0.04 mmol) and followed by levulinic acid (0.01 mL, 0.09 mmol). Finally DMAP (catalytic amount) was added. Then the reaction mixture was stirred at room temperature for 16 h. The progress of the reaction was monitored by TLC, starting material was disappeared. The reaction was diluted with DCM and the organic layer wash with water (10 mL×2 times), brine (10 mL) and organic layer was dried over Na₂SO₄, filtered and evaporated solvent under reduced pressure to get compound-7 (26 mg) as off white colored solid.

### 56SPL02211-03.

Stage-9:

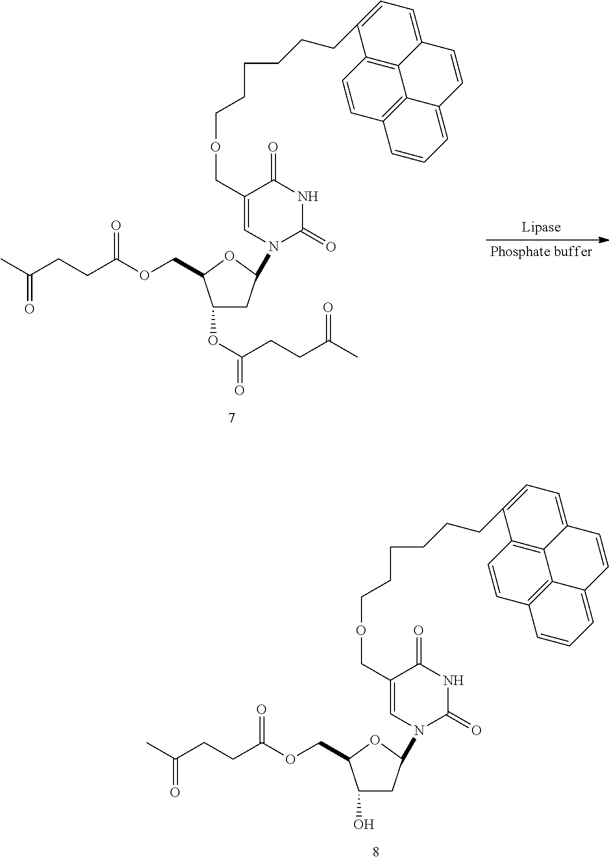 [SMILES: CC(=O)CCC(=O)OC[C@H]1O[C@@H](n2cc(COCCCCCCc3ccc4ccc5cccc6ccc3c4c56)c(=O)[nH]c2=O)C[C@@H]1OC(=O)CCC(C)=O ; CC(=O)CCC(=O)OC[C@H]1O[C@@H](n2cc(COCCCCCCc3ccc4ccc5cccc6ccc3c4c56)c(=O)[nH]c2=O)C[C@@H]1O]

### 5-methylhydroxy-pyrene-hexane-5′-O-lev 2′-deoxyuridine (8)

To a solution of compound-7 (0.2 mmol) in 1,4-dioxane (0.35 mL) is added 0.15 M phosphate buffer pH 7 (1.65 mL) and the lipase (CAL-A or PSL-C; 1:1 w/w). The mixture is shaken (250 rpm) for 6-10 hours while the reaction is monitored by TLC (10% MeOH/CH₂Cl₂). Upon completion of the selective hydrolysis of the 3′-O-levuninyl group, the enzyme is filtered and washed with CH₂Cl₂. The combined filtrates are concentrated and the residue after chromatographic purification furnishes compound 8 as white solid.
Reference: Garcia, J.; Fernandez, S.; Ferrero, M.; Sanghvi, Y. S.; Gotor, V. Building Blocks for the Solution Phase Synthesis of Oligonucleotides: Regioselective Hydrolysis of 3′,5′-Di-O-levulinylnucleosides Using an Enzymatic Approach. J. Org. Chem. (2002), 67, 4513-4519.

Stage-10:

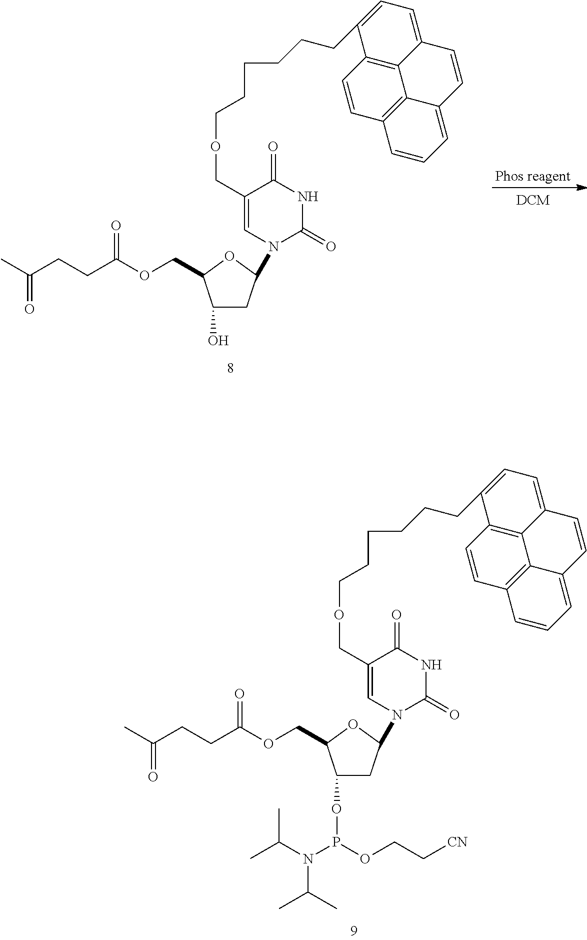 [SMILES: CC(=O)CCC(=O)OC[C@H]1O[C@@H](n2cc(COCCCCCCc3ccc4ccc5cccc6ccc3c4c56)c(=O)[nH]c2=O)C[C@@H]1O ; CC(=O)CCC(=O)OC[C@H]1O[C@@H](n2cc(COCCCCCCc3ccc4ccc5cccc6ccc3c4c56)c(=O)[nH]c2=O)C[C@@H]1OP(OCCC#N)N(C(C)C)C(C)C]

### 5-methylhydroxy-pyrene-hexane-5′-O-lev-2′-deoxyuridine-3′-O-amidite (9)

To a stirred solution of compound 8 (1 mmol) in dry CH₂Cl₂ (2.5 mL) is added the phosphorylating reagent (1.2 mmol) and the activator (Py.TFA or DCI; 1.2 mmol). The mixture is stirred for 1-3 hours while the reaction is monitored by TLC (10% MeOH/CH₂Cl₂). Upon completion of the phosphorylation, the solution is concentrated and the residue after chromatographic purification furnishes compound 9 as white solid.
Reference: Sanghvi, Y. S., Guo, Z., Pfundheller, H. M. and Converso, A. Improved Process for the Preparation of Nucleosidic Phosphoramidites Using a Safer and Cheaper Activator. Org. Process Res. Dev. 4, 175-181 (2000).

Stage-7:

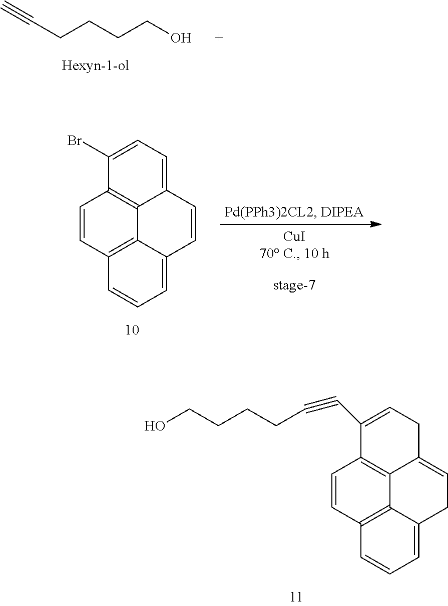

### Pyrene-hexyn-1-ol (11)

To a solution of compound-10 (10 g, 35.31 mmol) was dissolved in THF/Et₃N (600 mL 1:1), the solution was degassed by sparging with nitrogen for 30 min, then Pd (PPh₃)₂Cl₂ (1.2 g, 1.76 mmol), CuI (336 mg, 1.76 mmol) were added and degassed by sparging with nitrogen for 15 min, finally added hexyn-1-ol (11.7 mL, 105.94 mmol) and degassed by sparging with nitrogen for 10 min, a condenser was fitted to the flask, and the reaction flask was immersed into a preheated oil bath (80° C.). The reaction was allowed to proceed for 8 h and the solvents were removed in vacuum to give residue that was dissolved in EtoAc and given 1N HCl wash, water wash three times, finally brine wash. The organic layer was dried over Na₂SO₄, filtered and evaporated under reduced pressure. The crude compound was purified by silica gel (60-120 mesh) column chromatography, elute with EtOAc/Hexane (20-25%) to afford Pyrene-hexyn-1-ol as a light yellow solid [compound-11] (9.5 g, 90%).

### 33SPL02211-02.

Stage-8:

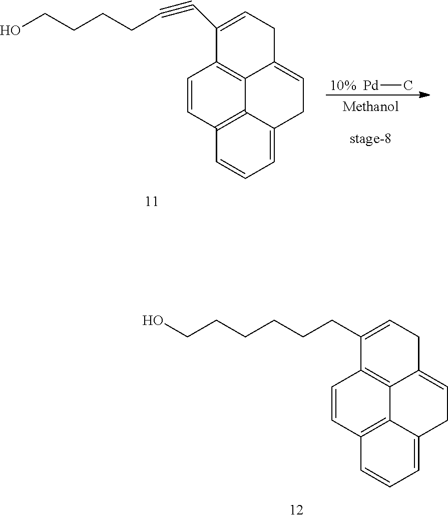

### Pyrene-hexanol (12)

Pyrene-hexyn-1-ol (10 g) was placed in a Parr bottle and dissolved in MeOH (300 mL) the container was flushed with nitrogen for 10 min. 10% Pd—C (1.2 g), was added. The reaction vessel was consecutively evacuated and pressurized with hydrogen two times eventually, then hydrogen pressure of 100 psi was maintained, and the suspension was shaken in the dark at room temperature for 16 h. The catalyst was removed by filtration through celite. The filtrate was concentrated under reduced pressure, and the residue was purification by column chromatography on silica gel (30% EtOAc in hexane) to yield Compound-12 (7.5 g, 74%) as an off white colored solid.

### 88SPL02211-02.

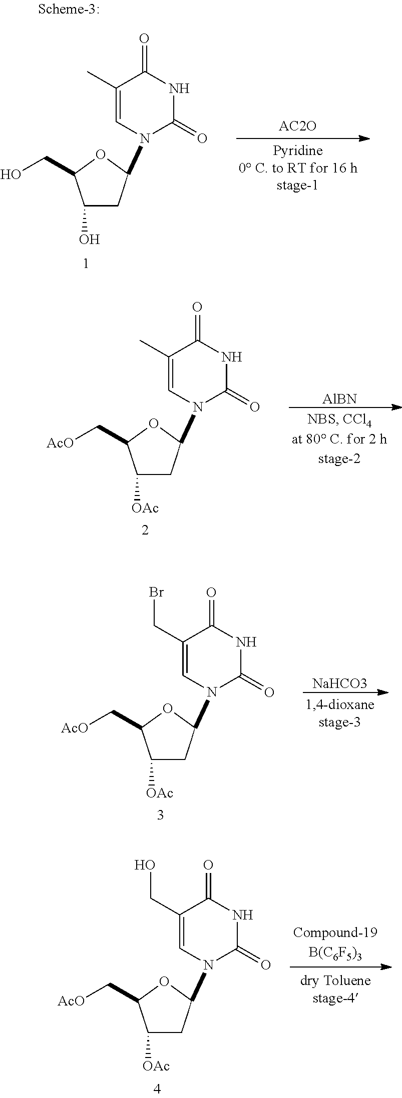

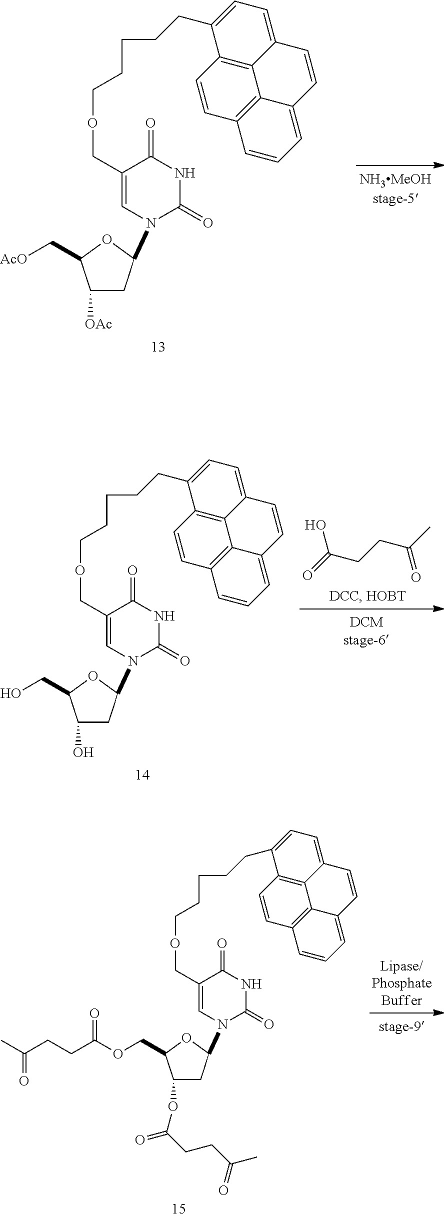

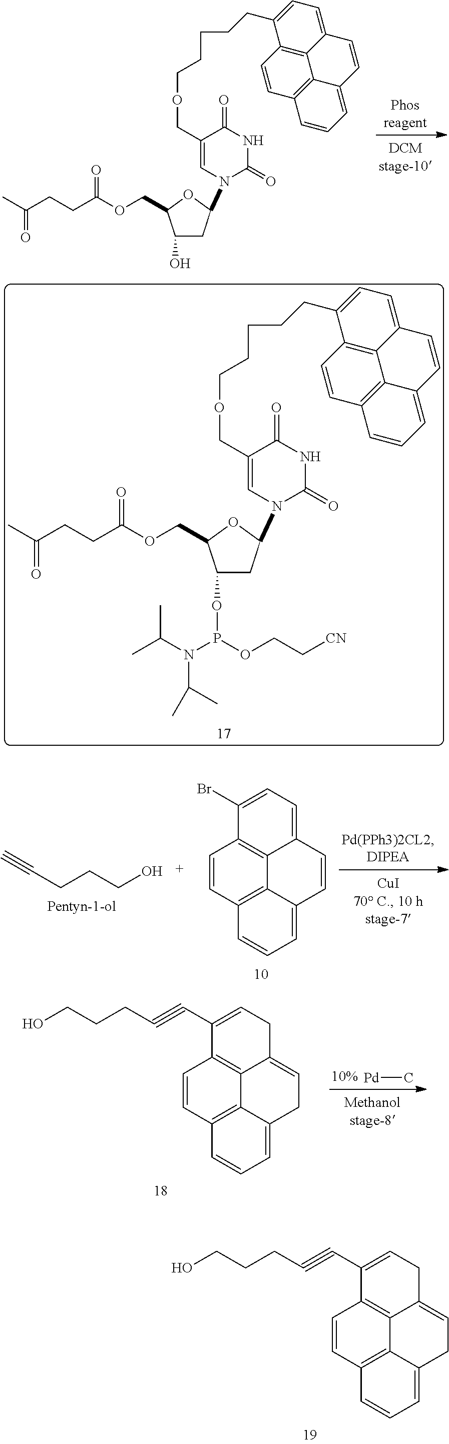

Please see the scheme-2, synthetic protocol up to compound-4.

Stage 4′:

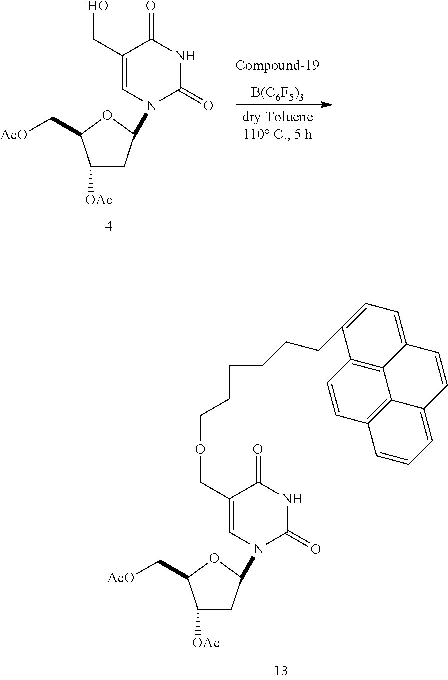 [SMILES: CC(=O)OC[C@H]1O[C@@H](n2cc(CO)c(=O)[nH]c2=O)C[C@@H]1OC(C)=O ; "B(C6F5)3" ; "Compound-19" ; CC(=O)OC[C@H]1O[C@@H](n2cc(COCCCCCCc3ccc4ccc5cccc6ccc3c4c56)c(=O)[nH]c2=O)C[C@@H]1OC(C)=O]

### 5-Hydroxymethyl-pyrene-pantane-5′,3′-O-Diacetyl-2′-deoxyuridine (13)

To a suspension of compound-4 (5.0 g, 14.61 mmol) and compound-19 (3.4 g, 11.69 mmol) in dry toluene at room temperature, then B(C₆F₅)₃ (748 mg, 1.46 mmol) was added to the reaction mixture under nitrogen atmosphere, Then the mixture was refluxed at 110° C. for 5 hrs. The progress of the reaction was monitored by TLC, starting material was disappeared. Then reaction mixture was cool to room temperature and evaporated under reduced pressure. The crude compound was dissolved with water (50 mL) and ethyl acetate (50 mL) and organic layer was separated, aqueous layer was extracted with EtOAc (25 mL×2 times), combined organic layers was wash with water (20 mL), brine (25 mL), dried over anhydrous Na₂SO₄ and evaporated under reduced pressure. The viscous liquid compound-13 (g) was taken for the next step.

### 47SPL02211-03.

Stage 5′:

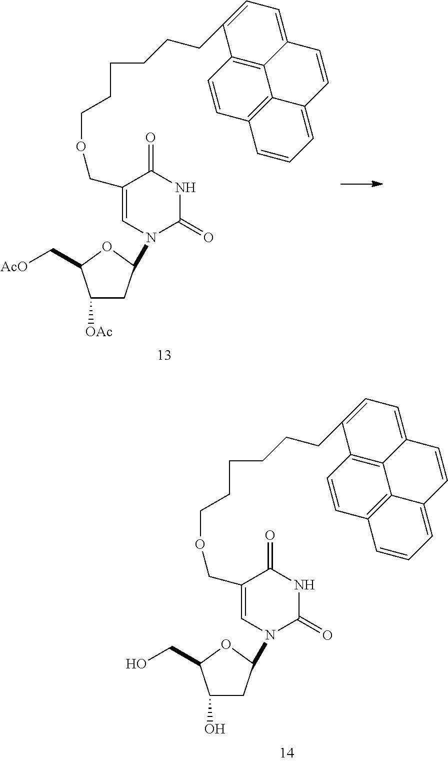 [SMILES: CC(=O)OC[C@H]1O[C@@H](n2cc(COCCCCCCc3ccc4ccc5cccc6ccc3c4c56)c(=O)[nH]c2=O)C[C@@H]1OC(C)=O ; O=c1[nH]c(=O)n([C@H]2C[C@H](O)[C@@H](CO)O2)cc1COCCCCCCc1ccc2ccc3cccc4ccc1c2c34]

### 5-Hydroxymethyl-pyrene-pentane-2′-deoxyuridine (14)

Compound-13 (2.0 g) was dissolved in 30 mL of MeOH.NH₃ solution, and stirred at room temperature for 16 h. The solvent was evaporated under reduced pressure, and the crude compound was diluted with EtOAc (30 mL), the organic layer was wash with water (15 mL), brine (15 mL), dried over anhydrous Na₂SO₄ and evaporated under reduced pressure. The crude compound was purified by silica gel (60-120 mesh) column chromatography elate with 5% MeOH in DCM to get compound 14 (200 mg) of off white solid compound.

Stage-6′:

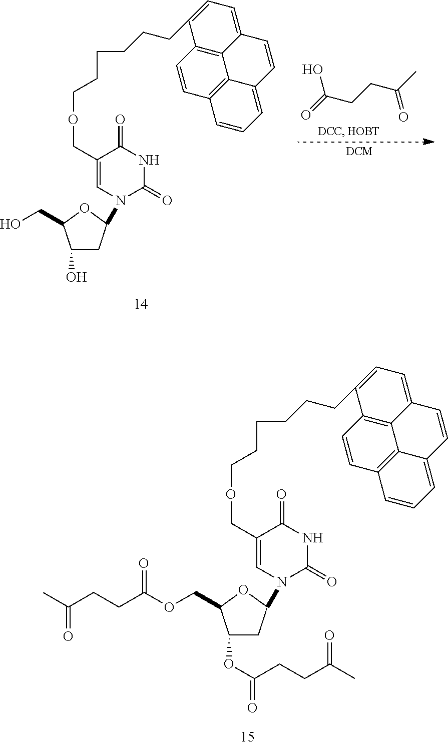

### 5-Hydroxymethyl-pyrene-pentane-5′,3′-O-lev 2′-deoxyuridine (15)

Compound-14 (25 mg, 0.046 mmol) is dissolved in dry DCM under nitrogen atmosphere, and stirred at 0° C. Then DCC (11 mg, 0.05 mmol), HOBt (6 mg, 0.05 mmol) and levulinic acid (0.01 mL, 0.09 mmol) are added sequentially. Finally DMAP (cat) is added. Then the reaction mixture is stirred at room temperature for 16 h. The progress of the reaction is monitored by TLC, starting material disappears. The reaction is diluted with DCM and the organic layer washed with water (10 mL×2 times), brine (10 mL) and organic layer is dried over Na₂SO₄, filtration and evaporation of the solvent under reduced pressure, furnishes compound-15 (26 mg) as off white colored solid.
Reference: Garcia, J.; Fernandez, S.; Ferrero, M.; Sanghvi, Y. S.; Gotor, V. Building Blocks for the Solution Phase Synthesis of Oligonucleotides: Regioselective Hydrolysis of 3′,5′-Di-O-levulinylnucleosides Using an Enzymatic Approach. J. Org. Chem. (2002), 67, 4513-4519.

Stage-7′:

### Pyrene-pentyn-1-ol (18)

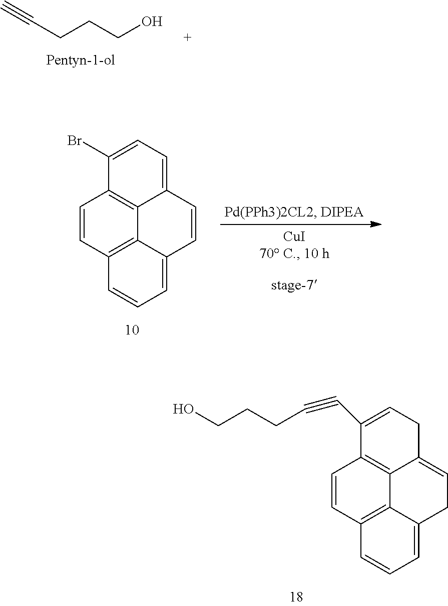

To a solution of compound-10 (10 g, 35.316 mmol) was dissolved in THF/Et₃N (600 mL 1:1), the solution was degassed by sparging septum with nitrogen for 30 min, then Pd (PPh₃)₂Cl₂ (1.2 g, 1.76 mmol), CuI (336 mg, 1.76 mmol) were added and degassed by sparging septum with nitrogen for 15 min, finally added pentyn-1-ol (9.8 mL, 105.94 mmol) and degassed by sparging with nitrogen for 10 min, a condenser was fitted to the flask, and the reaction flask was immersed into a preheated oil bath (80° C.). The reaction was allowed to proceed for 8 h and the solvents were removed in vacuum to give residue that was dissolved in EtoAc and given 1N HCl wash, water wash three times, finally brine wash. The organic layer dried over Na₂SO₄, filtered and evaporated under reduced pressure. The crude compound was purified by silica gel (60-120 mesh) column chromatography, elute with EtoAc/Hexane (20-25%) afforded compound-18 (9 g, 90%) as a light yellow solid.

### 34SPL02211-02.

Stage-8′:

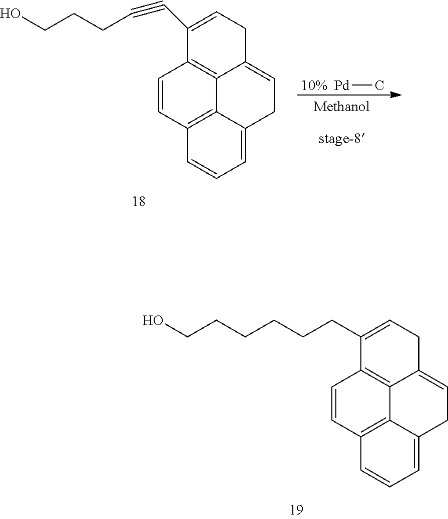

### Pyrene-pentanol (19)

Compound-18 (8.6 g) was placed in a Parr bottle and dissolved in MeOH (250 mL) the container was flushed with nitrogen for 10 min. 10% Pd—C (900 mg), was added. The reaction vessel was consecutively evacuated and pressurized with hydrogen two times eventually, then hydrogen pressure of 100 psi was maintained, and the suspension was shaken in the dark at room temperature for 16 h. The catalyst was removed by filtration through celite. The filtrate was concentrated under reduced pressure, and the residue was purification by column chromatography on silica gel (30% EtoAc in hexane) to get compound-19 (6 g, 69%) as an off white colored solid compound.

### 90SPL02211-02.

### REFERENCES

Ahmadian and Donald E. Bergstrom 2008, “5-Substituted Nucleosides in Biochemistry and Biotechnology.” In Modified Nucleosides in Biochemistry, Biotechnoloy and Medicine, P. Herdewijn, ed. Wiley-VCH, Weihheim, 2008, pp 251-276.
A K Todd, A Adams, J H Thorpe, W A Denny, L P G Wakelin and C J Cardin, J. Med. Chem. 1999, 42, 536-540.
Garcia, J.; Fernandez, S.; Ferrero, M.; Sanghvi, Y. S.; Gotor, V. Building Blocks for the Solution Phase Synthesis of Oligonucleotides: Regioselective Hydrolysis of 3′,5′-Di-O-levulinylnucleosides Using an Enzymatic Approach. J. Org. Chem. (2002), 67, 4513-4519.
K Sonogashira, Y Tohda and N Hagishara, Tetrahedron Lett. 1975, 16, 4467-4470.
M S Motawia, A E-S Abdel-Megied, E B Pedersen, C M Nielsen and P Ebbesen, Acta Chem. Scand. 1992, 46, 77-81; A E-S Abdel-Megied, E B Pedersen and C Nielsen, Monatshefte Chem. 1998, 129, 99-109.
Sanghvi, Y. S., Guo, Z., Pfundheller, H. M. and Converso, A. Improved Process for the Preparation of Nucleosidic Phosphoramidites Using a Safer and Cheaper Activator. Org. Process Res. Dev. 4, 175-181 (2000).
V V Filichev and E B Pedersen, J. Am. Chem. Soc. 2005, 127, 14849-14858; V V Filichev, I V Astakhova, A D Malakhov, V A Korshun and E B Pedersen, Nucl Acids Symp. Ser. 2008, 52, 347-348.

1. A modified oligonucleotide monomer SNA with the general structure:
X—B-L-I
wherein
X is a backbone monomer unit that can be incorporated into the backbone of an oligonucleotide or an oligonucleotide analogue,
B is a nucleobase, a pyrimidine or purine analog or a heterocyclic system containing one or more nitrogen atoms
L is a linker and
I is an intercalator comprising at least one essentially flat conjugated system and wherein the length of linker is between 5 and 15 angstroms.

2. The monomer of claim 1 further comprising a conjugator K between B and L or between L and I:
X—B—K-L-I
X—B-L-K—I

3. The X—B-L-I monomer of claim 1 being described by
X—B—CH₂O(CH₂)ₙ—I
wherein n is 5 or 6.

4. The X—B—K-L-I monomer of claim 2 being described by
X—B—K—(CH₂)ₙNHCO(CH₂)ₘCO—I,
wherein n is between 1 and 3 and m is between 1 and 3

5. The X—B-L-K—I monomer of claim 2 being described by
X—B—(CH₂)ₘ—O—(CH2-)ₙ—K—I
wherein m is 1 and n is 3 or 4

6. The monomer of claims 2, 4 and 5, wherein K is ethynyl

7. The SNA monomer of any of the preceding claims, wherein X—B is either a DNA or RNA unit.

8. The SNA monomer of any of claims 1-7 adapted for enzymatic incorporation into an oligonucleotide.

9. The SNA monomer of any of claims 1-7 adapted for incorporation into an oligonucleotide using standard oligonucleotide synthesis

10. An oligonucleotide comprising the SNA monomer of any of claims 1-7.

11. Use of the SNA monomer adapted for enzymatic incorporation of claim 8 as substrate for a polymerase.

12. Use of the oligonucleotide comprising the SNA monomer of claim 10 as primer or template in a polymerase chain reaction (PCR).

13. A method comprising the steps of
a. Providing a template nucleic acid
b. Providing a first primer oligonucleotide
c. Providing a polymerase
d. Providing a nucleotide triphosphate mixture
e. Mixing the components of steps a-d and providing conditions that allow the primer to anneal to the template.
f. Under conditions allowing primer extension, extending the first primer oligonucleotide annealed to the template
wherein the first primer oligonucleotide comprise a SNA monomer and/or
wherein the template nucleic acid comprise a SNA monomer and/or
wherein the nucleotide triphosphate mixture comprise a SNA monomer adapted for enzymatic incorporation into an oligonucleotide

14. The method of claim 13 further comprising the steps of
g. Providing a second primer oligonucleotide, which is complementary to the first extension product of step f
h. Denaturing the product of the step f
i. Under conditions allowing primer extension, extending the second primer oligonucleotide annealed to the first extension product
